(19) United States
(12) Patent Application Publication
(10) Pub. No.: US 2023/0112780 A1
(43) Pub. Date: Apr. 13, 2023

(54) METHOD FOR ANALYZING QUALITATIVE REMOTE USER EXPERIENCE AND USABILITY TEST RESULTS USING ARTIFICIAL INTELLIGENCE

(71) Applicant: ALEJANDRO RIVAS-MICOUD, MIAMI, FL (US)

(72) Inventor: ALEJANDRO RIVAS-MICOUD, MIAMI, FL (US)

(21) Appl. No.: 17/961,997

(22) Filed: Oct. 7, 2022

Related U.S. Application Data
(60) Provisional application No. 63/253,159, filed on Oct. 7, 2021.

(51) Int. Cl. G06N 5/02; G06V 40/20; G06V 20/40; G06V 40/16; G06T 7/20; A61B 5/16

(52) U.S. Cl. CPC G06N 5/022; A61B 5/165; G06T 7/20; G06V 20/41; G06V 20/44; G06V 40/20; G06V 40/174; G11B 27/34

(57) ABSTRACT

A method for analyzing qualitative remote user experience and usability test results using artificial intelligence. At least one participant is selected to interact with a test session through a remote testing software. Data is recorded from the test session and inputted into a central computer for data analysis. Moments of interests of the participant’s interaction are identified by synthesizing semantic data, eye tracking data, biosensor input data, and facial analysis from the inputted recorded data. The artificial intelligence system is trained classifying an identified moment of interest as a detracting event, classifying a non-identified moment of interest as a moment of interest, identifying which input data is associated with the detracting events, identifying which input data is associated with the non-identified moments of interest, and identifying which input data is associated with moments of interest. The recorded data is outputted with the identified moments of interest.

### CROSS REFERENCE TO RELATED APPLICATION

This application claims the benefit, under 35 U.S.C. § 119(e), of Provisional Patent Application No. 63/253,159, filed Oct. 7, 2021; the prior application is herewith incorporated by reference in its entirety.

### FIELD AND BACKGROUND OF THE INVENTION

The present invention relates to a method for analyzing qualitative remote user experience and usability test results using artificial intelligence. The user experience is an area of strong and growing interest for companies and organizations, as organizations that have designed and implemented a superior user experience and usability achieve a high degree of success, in terms of increased market share, lower rates of churn/loss of clients, lower expenses related to customer service, higher pricing power, significant competitiveness and higher profitability.

Usability and user experience (UX) research and testing (complementary disciplines with a common goal) create valuable insight for organizations, allowing entities to know: at what times, in what aspects, and in what way users and customers feel frustrated and/or lack knowledge in achieving their goals while interacting with digital assets (e.g., web pages, mobile applications, prototypes, among others). In this sense, organizations dedicate many efforts and resources towards understanding and comprehending the user experience to transform and optimize their assets. Ultimately, the ease of use in all digital interfaces guarantees a successful product through the pleasure of the target audience.

A simple illustration highlights why the user experience of a digital asset is particularly important; if a user enters a physical store or restaurant, and is not pleased with the experience, there is a certain amount of embedded inertia that must be overcome before they decide to leave, and try a different store or restaurant.

However, in the digital world, as soon as a user detects an inferior usability or user experience, they will click away to an alternative within seconds. This directly portrays the stakes in “getting it right” are far higher, in relation to the usability and user experience of digital assets, vis-a-vis physical locations.

User experience research is one of the most necessary activities in any company. In the past decades, a number of companies and platforms have emerged that have reduced what used to be weeks or even months in terms of the time required to launch a qualitative (i.e., with recordings of subjects interacting with a digital asset) study and receive results, to days or even hours.

However, a key challenge for companies and organizations is scalability. In other words, as the need for user experience research grows exponentially within organizations, the ability for the user experience research departments to analyze the growing volume of qualitative user experience research results is impeded by a lack of resources and time; whereas quantitative market research or usability or user experience studies can be analyzed quickly, through reviewing charts that illustrate the aggregate results of hundreds or even thousands of respondents. This is not the case with qualitative studies (i.e., recorded sessions); each respondent session may be anywhere from 15 to 60 minutes long, so if a user experience researcher launches a study, for example with 30 respondents, with an average of 30 minutes each session, said researcher will need to review 15 hours of video to extract conclusions from each such study they launch.

The purpose of the invention, described later on, is to leverage artificial intelligence and a series of inputs so as to quickly identify which timeline moments of each session recording of each study merit a deeper evaluation and analysis; thus saving a significant amount of time, and allowing for a scaling up of user experience testing and research to accommodate the growing and explosive demand being experienced in this sector.

Userlytics has allowed companies to establish user experience tests anywhere in the world, through the Userlytics remote platform. The platform (www.userlytics.com) enables the recording of a combined “Picture-in-Picture” audiovisual recording of participants (i.e., what they see and do, as well as they themselves), through a video recording of their screen as they interact with digital assets (i.e., websites, mobile apps, prototypes and other digital assets), by following instructions, while they speak out loud in a “Think Aloud” protocol, and also captures and allows the manipulation and sharing of a number of additional qualitative and quantitative metrics of each user experience and usability test. Userlytics Client’s desire Userlytics’ services for a number of purposes, such as evaluating a product’s functions, features, and purposes in line with the User’s desires. Clients observe realistic or actual user interactions through the testing platform; this observance allows the client to learn user behavior, evaluating the needs and expectations of the user while creating or developing their user interfaces.

The “Return on Investment” (ROI) experienced by companies that have focused on improving the experience of their users and clients has led to a significant increase in the content created through User experience testing videos. As a result, the markets have experienced exponential growth, especially over the last decade, include, but not limited to, the United States, United Kingdom, Canada, Australia, New Zealand, Netherlands, and Scandinavia, among others.

Remote user tests are a powerful online research tool based on the methodology of analysis by user objectives or scenarios, which allows testing the usability and user experience through sophisticated remote testing software. However, a significant amount of qualitative (i.e., video) content is created as the tests are conducted, fueling problems for user experience (“UX”) researchers who allocate resources and time towards the manual review of all content derived from the qualitative UX testing. The ever-increasing supply of UX testing videos, full of data and powerful “insights” to increase and improve user experience and usability, requires time and resource allocation for video and analysis review.

Researchers evaluate qualitative variables through different tests that reveal how users interact with the product, and large volumes of data are collected. Data, especially audiovisual data, is used by companies to understand, mainly, how users use a product, what frustrates users, what users do not understand, how and why users fail to realize a usage goal and the like. This data analysis is essential to optimize the user experience while remaining fundamental and critical for any organization that creates, develops, and maintains digital interfaces.

The aforementioned tests involve an astute and orderly investigation of components, including, but not limited to, the organization’s function, product or service, sector, and target audience, among others. The tests, and thus, analysis, generally include the following stages:
1) definition of objectives;
2) decision on the type of test/questionnaire;
3) provision of the digital asset to be tested;
4) identification of the types of user profiles;
5) design of tasks and questions;
6) selection and recruitment of users;
7) carrying out the test; and
8) analysis of results.

Despite the heterogeneity in the content, the main difficulties lie in analyzing qualitative results (i.e., audiovisual). Analyzing large volumes of audiovisual content quickly and effectively is a task that has proven to be most complex and time-consuming. Thus, generating more relevant and exciting conclusions (“insights”) in a timely and scalable manner is difficult, negatively affecting the optimization of the user experience.

For example, in UX studies, nonverbal behavior may convey more information than verbal behavior. In other words, in user experience studies, we find that what people do, is oftentimes different than what people say they did, or what they would do, and thus, the audiovisual recording of what they actually do is essential. Therefore, it is necessary to study behaviors parallel to speech that transmit useful information to outline strategies to maximize the insight captured from the said UX studies. This type of communication is analyzed by inspecting the video recordings (i.e., sound and image) of each individual participating in the test.

Since tests can vary from five to thousands of people, a manual analysis is highly inefficient and, in some cases, impossible to conduct in a cost-effective and streamlined way. Currently, some products favor the work of sound and image analysis; however, the focus on these aspects does not offer more than twenty percent of the data that is needed to conduct a proper study. Nevertheless, software is available for data collection, such as sound analysis, transcription of audio to text, and image analysis and/or eye tracking.

Audio-to-text transcription systems make it possible to collect the verbal content of audio recordings; these types of tools speed the work of analysts, collecting people’s conversations in text format; examples of these tools include Voicebase and Amazon Transcribe.

Revising and analyzing text has proved difficult; although the range of available languages has expanded, the transcription systems do not offer features that simplify or summarize the content, much less highlight the critical metrics or the data that requires the most dedication of effort on the part of the stakeholders. Furthermore, language analysis (“semantic analysis”) goes beyond the simple “sentiment analysis” (i.e. joy, sadness, neutral) to extract segments of each video automatically.

“Eye tracking” technology focuses on the direction of the gaze; this technology monitors the eye tracking by evaluating movements relative to a surface (i.e., screen, object, and other variables). The moments in which the gaze remains longer at a certain point or at points where the eyes move from side to side quickly or slowly provide information about the printing or layout of the content on new products or displays.

Some eye tracking tools integrate the use of webcams and software, without the need for the traditional eye tracking lab hardware, to achieve the same result. However, the true potential is derived from the unification of eye tracking (i.e., “where am I looking”), semantic analysis (i.e., “what am I saying at that moment and what state of mind are indicated by the words and phrases used”), sensory data from “wearables,” and gesture and facial expression analysis.

Automatically integrating the data with artificial intelligence, complemented by a machine learning system fed by human expert analysis to “correct” mistakes of the AI system allows organizations to identify which moments of the video content are most interesting to study, paradigmatically decreasing the time needed to analyze each user experience study or qualitative market research.

The spectacular growth in demand for testing and analysis is driven by the accelerating recognition of the need for an optimization of the user experience. This present invention massively cuts the time and resources necessary for the analysis of the explosive growth in qualitative user experience video results.

The present invention relates to a system for automating the identification of critical events in audiovisual recordings. The invention further relates to the incorporation of artificial intelligence, machine learning, semantic analysis, eye tracking, biosensor-based feedback, and facial analysis to automatically identify, from a specific video and specific timeline moment perspective, the critical events of audiovisual recordings.

The system allows stakeholders to illustrate the specific events that would be considered “interesting” for said user. The system is programmed to extract interesting moments by integrating different types of criteria. For example, the criterias include: (i) general criteria (e.g., the types of events that would be considered of interest in an audiovisual recording of a market research study or usability test), (ii) specific sector criteria (e.g., the same, but applied to a specific industrial sector like finance, or biotechnology), as well as (iii) customized criteria (e.g., user “programs” the system to illustrate the specific types of events and comments that would be considered “interesting” for said user).

Completing and analyzing the results of a study can range from weeks to even months, with the current manual system for reviewing each and every video.

Suppose a single UX researcher, on average, launches one qualitative test per week, of 10-15 participants, with 30 to 60 minutes of recording time each. In that case, this implies a total “review time” necessary to identify the key moments of each video, of between five hours and fifteen hours per week, at minimum (not taking into account the time needed to rewind, confirm, etc.). In other words, the total time for each user to carry out this type of work can approach between 12.5 and 37.5% of the total work week of said researcher. Individuals working in marketing, design, and similar departments also may review the audiovisual recordings to, ultimately, extract insight on their customers. Eminently possible with the AI and machine learning based system’s initial detection of key moments, organizations will save substantial time by reducing the resources needed for content review.

U.S. Pat. No. US 5414644 A, titled Repetitive Event Analysis System and filed by Ethnographics, Inc., shows one example of an automation process by integrating software to observe and compare visual records.

Another example is present in U.S. Publication No. US 20050188328 A1, titled Audiovisual information management system with presentation service.

There are several platforms that, based on the use of “semantic analysis,” try to identify, automatically, moments in which a person (either a participant in a market research study or a person interacting in a social network) demonstrates positive or negative feelings (commonly referred to as “sentiment analysis”). However, these systems rely on a simple analysis of phrases and words.

Facial recognition has experienced a very considerable advance in recent years, allowing the most advanced systems to identify, with pinpoint accuracy, the identity of people. Even so, with some precision, the identification of feelings or moods is still very incipient in facial analysis systems.

“Eye Tracking” systems, which track where a person’s gaze is or is headed, have recently experienced many advances. Most recently, advances in the quality of webcams and software have enabled “software only” eye tracking systems rather than the hardware-based eye tracking systems commonly used.

These technologies have commonly been used in marketing analysis and usability testing cases. However, the technology has not been used to date as part of a holistic analysis of audiovisual recordings to automatically detect key events and comments (“Moments of Interest”, MOI). The following technology creates an efficient system for Clients to identify MOI in the qualitative data created via the qualitative UX testing environment.

### SUMMARY OF THE INVENTION

It is, therefore, an object of the present invention to solve the aforementioned problems in the art by automating the identification of key moments in any audiovisual video recording, regardless of the use case of the recording (i.e., market research, usability testing, marketing, among others) and using AI to further increase efficiency and reliability on the identification of key moments.

This object comprises a system for combining a series of data inputs extracted from the testing environment and then applying algorithms based on artificial intelligence and continued data input and corrections through machine learning to improve the system’s accuracy. The data inputs may vary depending on the project’s specification, which the Client ultimately decides based on the desired testing results. For example, clients can conduct remote studies with the target customer in an interview-like setting to gather a myriad of verbal and nonverbal data; this data may be coupled with visual data through a moderated, recorded study.

This object also merges and expands upon the incipient technology in the art; the present invention allows for a contextual intermingling of each of the data points relevant to the key events in user experience testing—each data input adds context to the others. With the additional context and layers of input brought to the analysis by the present invention’s system, results are provided with increased accuracy.

In a preferred embodiment, data is extracted from a test session utilizing different technologies. For example, analyzing the voice from the tester in order to detect positive, negative or neutral sentiments; analyzing biometrics insights using wearables collecting the pulse or other data; tracking the eyes so the system detects the areas where the users focus more, among others.

The data points from the project are merged and therefore unified from the multiple sources, integrated in the artificial intelligence based algorithm to create the desired output for the client.

Data is collected from the external services through public Application Programming Interfaces (API) in JSON and/or XML format. Userlytics servers then communicate with the external APIs to gather the information, and/or the external services connect with Userlytics servers to provide the information. Several databases host different types of data; for example, sentiment analysis, biometric analysis, and eye tracking result in different databases.

Once the external information is collected, it’s processed and saved in the databases. Depending on the nature of the data, NoSQL databases work better than traditional SQL databases. The AI application is able to access all databases to collect all the information and feed the algorithm. This way, the AI can create the predictions from the previous results and analysis.

This contextual analysis can be used beyond user experience testing, as the technology applies to other sectors and industries. Utilizing a holistic integration of different inputs (i.e., eye tracking, semantic analysis of transcribed audio, automated facial analysis, biosensor inputs, among others) through artificial intelligence and machine learning, the technology can be used in any medium whereby a user desires to automatically identify the key events, actions and comments of interest (All, MOI) of an audiovisual recording.

Many of these technologies have been individually used in marketing analysis and usability testing cases. However, they have not been used to date as part of a holistic analysis of audiovisual recordings to automatically detect moments of interest (MOI), i.e.: actions, comments or events that a user experience researcher would normally wish to see and hear and evaluate as part of their analysis..

With the foregoing and other objects in view there is provided, in accordance with the invention, a method for analyzing qualitative remote user experience and usability test results using artificial intelligence, the method comprising the steps of:
selecting at least one participant based on predetermined criteria;
recording data of the at least one participant’s interaction with a test session through remote testing software;
inputting the recorded data from the test session into a central computer for data analysis;
identifying a plurality of moments of interests of the participant’s interaction from the test session by synthesizing semantic data, eye tracking data, biosensor input data, and facial analysis from the inputted recorded data;
training the artificial intelligence by
   classifying at least one identified moment of interest as a detracting event,
   classifying at least one non-identified moment of interest as a moment of interest,
   identifying which input data is associated with the detracting events,
   identifying which input data is associated with the non-identified moments of interest, and
   identifying which input data is associated with moments of interest, outputting the recorded data of the participant’s interaction with the test session with the identified moments of interest.

In this embodiment certain inputted data is associated with either detracting events, non-identified moments of interest, and moments of interest. This identification of data is fed into the AI for learning. In further iterations of identifying moments of interest, this data impacts the identification with consideration of their association with detracting events, non-identified moments of interest, and moments of interest.

The software-based method for analyzing qualitative (video based) remote user experience and usability test results creates advantages by establishing, based on a specific or general criteria, participants to provide data and information as part of a study; collecting the data from hardware and software, or the intermingling of computer systems, which are used by participants during the testing process; extracting the data derived from the testing environment for data processing activities; automatic integration of all the data sets into a system for data analysis; synthesizing the computer systems, and qualitative and quantitative inputs, such as voice responses, biometric data and visual information by capturing: semantic data of the machine based voice transcriptions from the user involved in the audiovisual recording established via the UX testing environment, including a sentiment analysis of said transcriptions; eye tracking data, so as to incorporate the direction of the eye and what is being viewed, with the semantic analysis of what is being said; biosensor input, so as to incorporate the pulse rate, and other sensed biofeedback, with the direction of the gaze and the semantic analysis of what is being said and the sentiment analysis; and facial analysis, so as to combine an analysis of the facial expressions with the direction of the gaze, bio sensor based feedback and semantic and sentiment analysis of what is said.

An added preferred development of the method includes wherein the recorded data is a video recording of the participant’s screen during the participant’s interaction with the test session, an audiovisual recording of the participant’s interaction with the test session, and/or biosensor data from a biosensor device worm by the participant during the test session.

An additional preferred development of the method is wherein the recorded data is outputted to a user interface with the video recording of the participant’s screen and/or the audiovisual recording of the participant having identified moments of interest timestamped.

A further preferred development of the method further comprises the steps of identifying data sets that are associated with multiple detracting events; identifying data sets that are associated with multiple non-identified moments of interest; and identifying data sets that are associated with multiple moments of interest.

In this embodiment certain combinations of inputted data, or data sets, are found to reoccur in either detracting events, non-identified moments of interest, and moments of interest. This identification of data sets is fed into the AI for further learning.

An additional preferred development of the method further comprises the steps of identifying data sets that are associated with at least one detracting event; identifying data sets that are associated with at least one non-identified moment of interest; and identifying data sets that are associated with at least one moment of interest.

In this embodiment certain combinations of inputted data, or data sets, are found to occur in either detracting events, non-identified moments of interest, and moments of interest. This identification of data sets is fed into the AI for further learning.

An added preferred development of the method is wherein the recorded data is input into the central computer during the at least one participant’s interaction with the test session; and the method further comprising the steps of identifying at least one moment of interest during the at least one participant’s interaction with the test session; training the artificial intelligence during the at least one participant’s interaction with the test session; and identifying, thereafter, at least one further moment of interest.

This further embodiment allows for the artificial intelligence to learn and inform the identification of moments of interest as the test session progresses. Data and analysis from the current test session can be used to increase reliably of further identified moments of interest or correct previously identified moments of interest.

Remote user experience testing platforms are a powerful online research tool based on the methodology of analysis by user objectives or scenarios, which allows for testing the usability and user experience of digital assets (websites, mobile apps and prototypes amongst others) through sophisticated remote UX testing software.

Further contemplated variations of the method are that testers can use a modern browser, that they may use an Overlay recorder, which is a browser extension; tests can use a mobile app, which includes an embedded browser, which may be used or not, based on the study configuration (it may use the native mobile browser). Presentation of the test further includes running at least one software program in charge of tracking the behavior, voice, image and biometric data. The method includes storing, using the one or more computer systems, the collected information in at least one database of the one or more computer systems. The data is aggregated, organized, and configured for artificial intelligence-based analysis, developing the natural language processing and knowledge engineering of the artificial intelligence system. The machine based transcription of the extracted data from the recording is analyzed automatically, and searched for keywords and key phrases that have been previously defined or added as a specific customization by a user, or incorporated and added by the AI algorithm and/or machine learning component. The analysis of biometric data, collected by wearable devices, is analyzed by the client and/or professional staff, and/or added by the AI algorithm and/or machine learning component. The analysis of gesture recognition data, collected by a specific device and/or a camera, is analyzed by the client and/or professional staff, and/or added by the AI algorithm and/or machine learning component. The analysis of eye tracking data, collected by a camera, is analyzed by the client and/or professional staff, and/or added by the AI algorithm and/or machine learning component.

The novel integration of this technology can be applied to analyze any type of audiovisual content in which one or more people are talking or answering questions about any topic, analyzing a website, application, prototype, or any type of audiovisual recording that includes a significant amount of commentary or dialogue. In the following description, for purposes of explanation, numerous specific details are set forth to provide a thorough understanding of the present invention.

The terminology used herein is for the purpose of describing particular embodiments only and is not intended to be limiting of the invention. As used herein, the term “and/or” includes all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The present disclosure is to be considered as an exemplification of the invention and is not intended to limit the invention to the specific embodiments illustrated by the figures or description below.

### BRIEF DESCRIPTION OF THE FIGURES

FIG. 1 shows a general overview of the system for automating the identification of key events of audiovisual recordings.

FIG. 2 shows a general overview of participant interaction with the testing platform via browser.

FIG. 3 shows the sentimental analysis process.

FIG. 4 shows the sentiment analysis interface composed of a video player and the transcription.

FIG. 5 shows an overview of biometric analysis.

FIG. 6 shows the device capturing facial expressions and timestamps once the user starts a session

FIG. 7 shows the device capturing eye movements and timestamps after the user starts a session.

FIG. 8 shows that the AI module is composed of two phases.

### DETAILED DESCRIPTION OF THE INVENTION

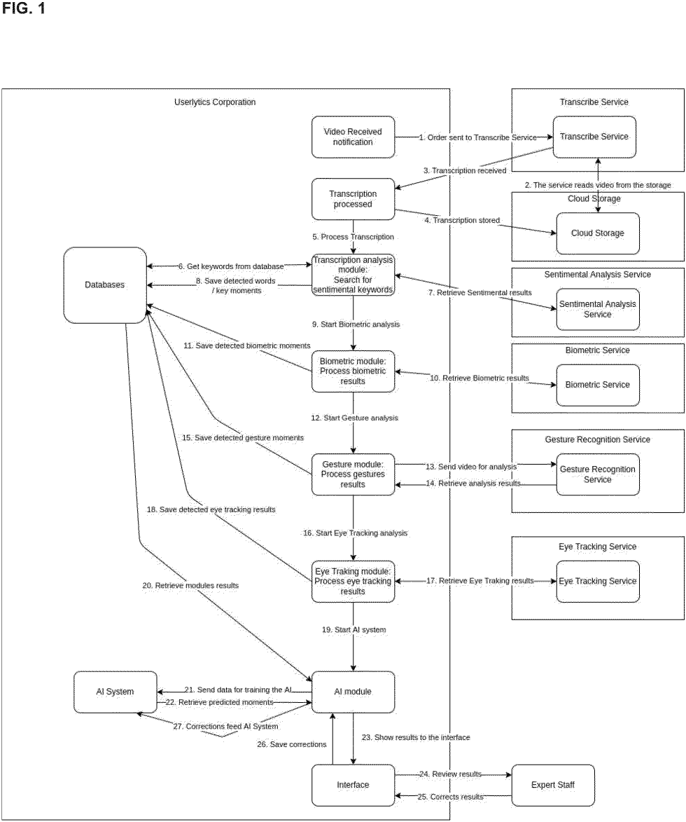

The present invention will now be described by referencing the appended figures representing preferred embodiments. FIG. 1 depicts the process with the integrated technology; through the incorporation of multiple inputs leveraging separate technologies with artificial intelligence, allowing users to efficiently produce and evaluate results.

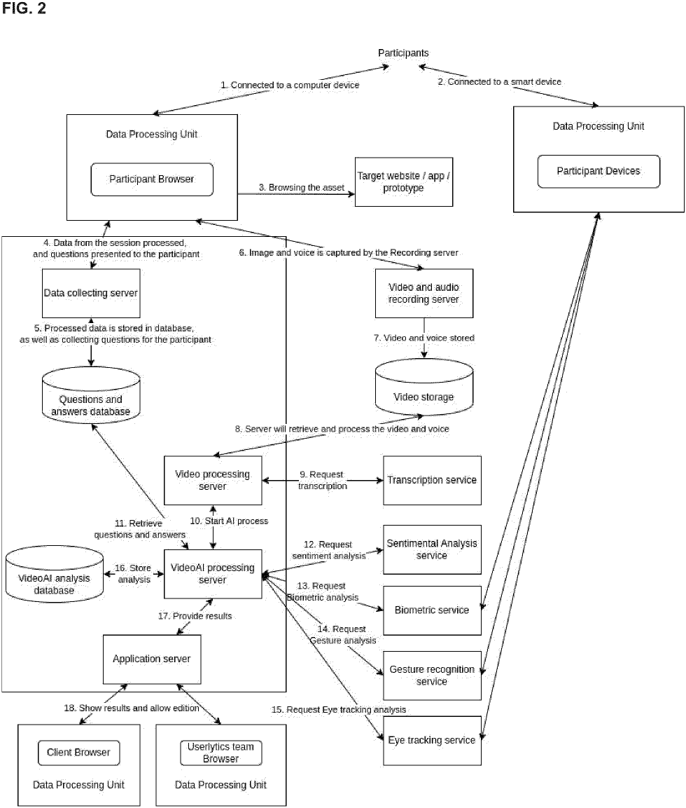

As shown in FIG. 2, the participants accessing a testing platform via Browser. The platform will redirect them to the target site, prototype, and application. While following the instructions, participants will be recorded, and the answers collected. At the same time, participants’ devices will collect extra information, such as but not limited to biometric information, gesture recognition, and eye tracking. Once a participant finishes the instructions, the video is processed by the platform, requesting an automatic transcription. When the transcription is available, the sentiment analysis process starts, followed by the rest of the processes (i.e., biometric, gesture recognition, eye tracking) to collect all the information, process it, feed the artificial intelligence, and then make all results available to the users.

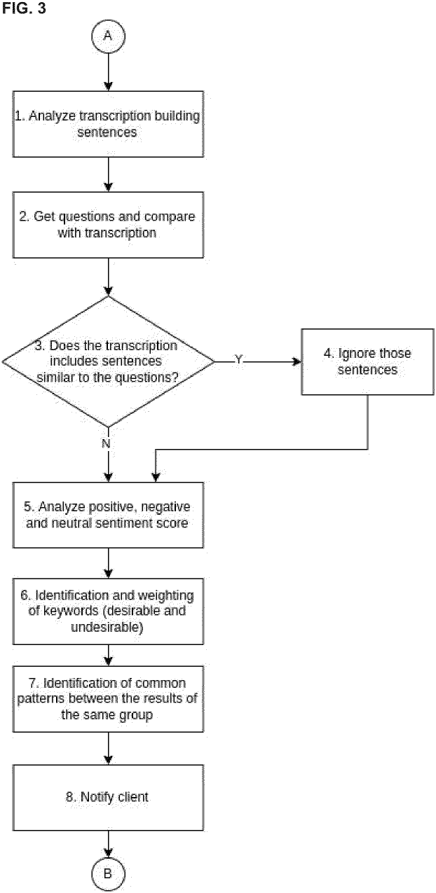

FIG. 3 shows the sentimental analysis process by following a series of steps as getting the study questions, participant answers, and transcription; comparing the transcription with the questions in order to discard them from the analysis; analyzing positive, neutral, and negative sentiments from the rest of the sentences; identifying keywords highlighting positive and negative sentences; identifying common patterns in the sentiments in the same study; and optionally, the client may be notified that the sentiment analysis results are available

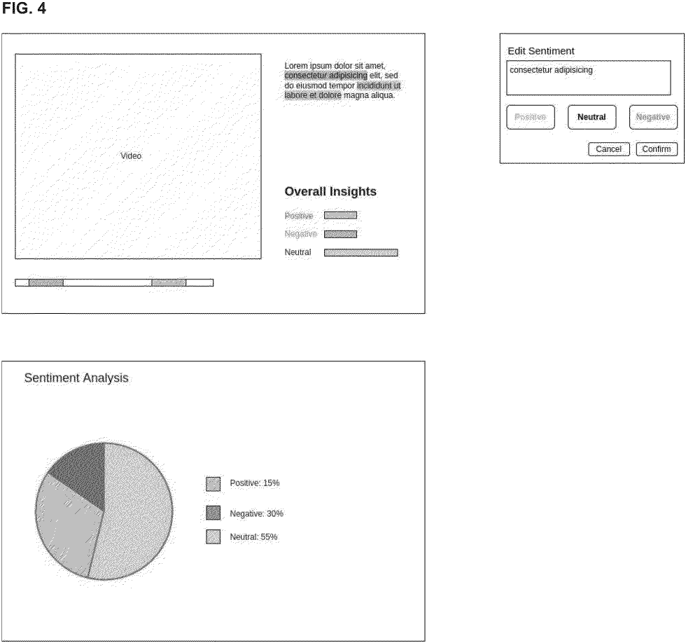

FIG. 4 shows the sentiment analysis interface composed of a video player and the transcription. The transcription sentences are highlighted in colors depending on the positive, neutral, or negative result. In addition, the video player includes a timeline adding the same colors for highlighting the moments when the session represents a positive, neutral or negative sentiment. In order to represent the results at a global level for one specific study, the chart shows the percentage of positive, neutral, and negative results among the participants.

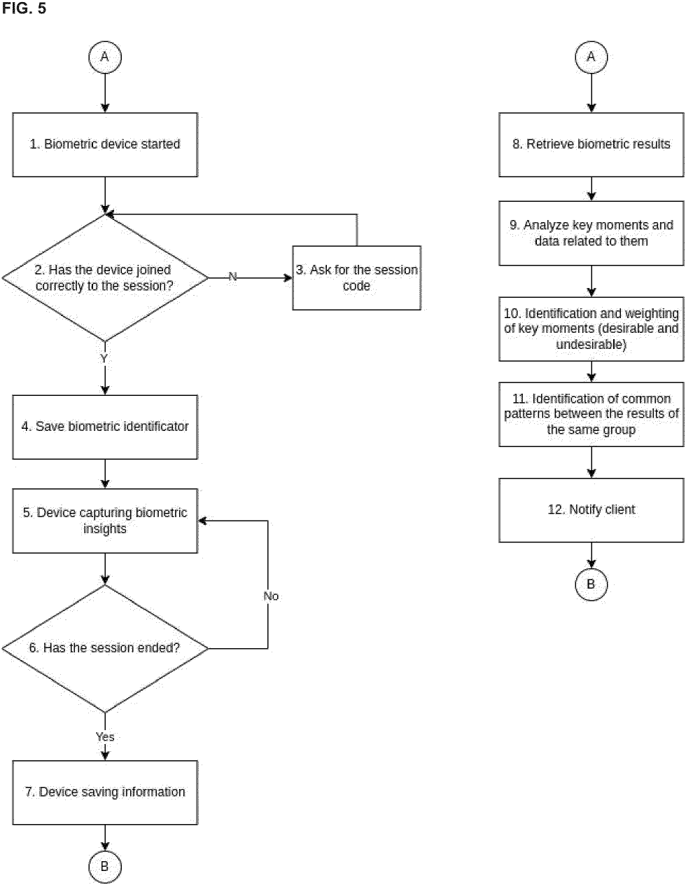

FIG. 5 shows the biometric analysis consisting of the device pairing, data collection, and data analysis. Device pairing and data collection. Data analysis. The pairing must link a session with the device and the data it collects. The analysis consists of selecting the desired data for processing and future references. Once the user starts a session, he/she links the device. While doing the test, the device captures data, such as the pulse, other parameters, and timestamps. The biometric analysis process follows a series of steps of getting the biometric data and timestamps; retrieving the key moments detected by the device; adjusting or detect false positives if required based on previous results and learning; identifying common patterns and feed the system for future usage; and optionally, the client may be notified that the analysis results are available

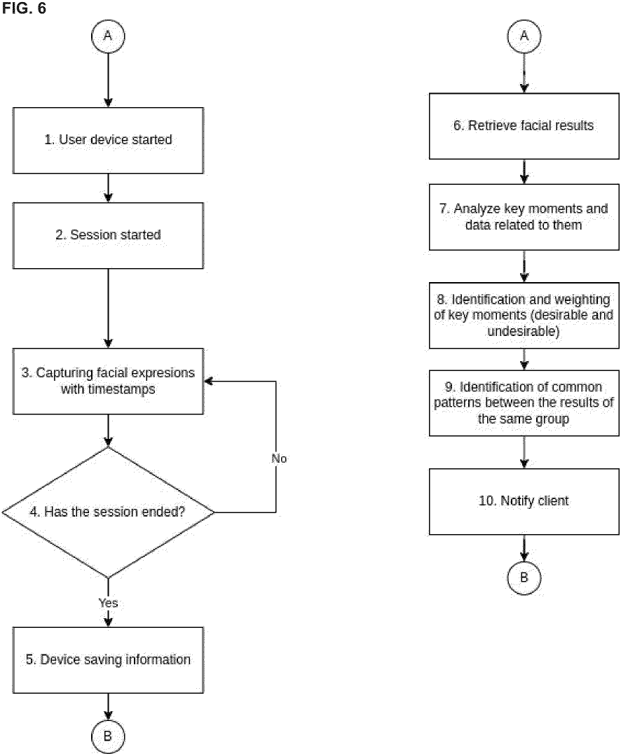

FIG. 6 shows the device capturing facial expressions and timestamps once the user starts a session. The result is a set of expressions (positive, negative, or neutral, as well as others) and their corresponding timestamps. The analysis process follows a series of steps of getting the facial expressions data and timestamps; retrieving the key moments detected by the device; adjusting or detecting false positives if required based on previous results and learning; and identifying common patterns and feed the system for future usage; and optionally, the client may be notified that the analysis results are available

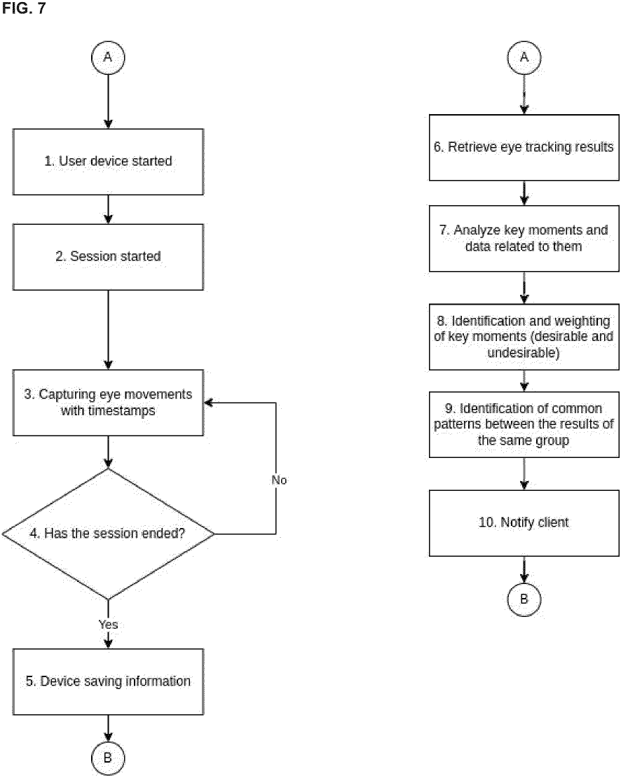

FIG. 7 shows the device capturing eye movements and timestamps after the user starts a session. If the user focuses on one specific screen area, it records the time spent on that area. The result is a set of locations and their corresponding timestamps. The analysis process follows a series of steps of getting the eye tracking data and timestamps; retrieving the key moments detected by the device; adjusting or detecting false positives if required based on previous results and learning; identifying common patterns and feed the system for future usage; and optionally, the client may be notified that the analysis results are available. The interface shows a heatmap based on a timeline where red areas represent the sections on which the user spent more time.

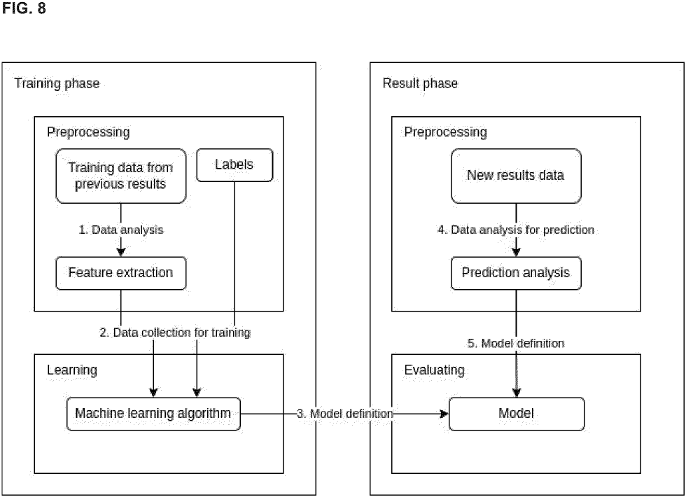

FIG. 8 shows the Training phase whereby previous results from other modules, such as Sentiment Analysis, and Biometric Analysis, will train the machine learning algorithm. The results will be classified and analyzed based on tags for future usage. The analysis generates data (key moments and metadata) to feed the model.

FIG. 8 shows the Prediction phase, whereby new results from the other modules will be analyzed to predict the key moments. Those pivotal moments are also included in the model for the subsequent prediction execution.

In a preferred embodiment, an audiovisual recording is made of a market research participants, users, usability testers, or user experience tester’s (All, “Tester’s”) interaction with the User Experience Testing Environment. From the audiovisual recording, data inputs related to the following are collected:
(i) Semantic analysis of machine-based voice transcriptions, using both user-defined words and phrases, as well as the results of continual iteration and feedback mechanisms to “teach” the system to improve its accuracy, in addition to “customizing” the system for a particular industry or goal;
(ii) Eye tracking, so as to incorporate the direction of the eye and what is being viewed, with the semantic analysis of what is being said;
(iii) Biosensor input, so as to incorporate the pulse rate, and other sensed biofeedback, with the direction of the gaze and the semantic analysis of what is being said; and
(iv) Facial analysis, so as to combine an analysis of the facial expressions with the direction of the gaze, bio sensor based feedback and semantic analysis of what is said.

The system incorporates these data inputs and optionally the goals and customizations defined by the user (e.g., industry sector-specific, or even company or project-specific goals as embodied by specific phrases or words) to identify by video and timeline moment the most interesting moments of the recordings. A manual review of the recordings can be compared with the automatic review to eliminate words, phrases, or content that is or is not considered interesting. Choosing the words, phrases, and content that are considered interesting automatically “teaches” the system through a continual and iterative feedback process, which increases the system’s overall accuracy through machine learning. This system can be used with a single tester interaction or audiovisual content in which one or more people are talking about any topic, answering questions, performing tasks while following a “think aloud” protocol, or analyzing a website or application, or digital prototype.

Although the invention is discussed in detail regarding and market research, usability testing, and user experience testing, the same system can be used for other mediums, such as police interrogations; journalist based interviews, and movie and advertising production, among others.

In a preferred embodiment, the system comprises five phases: transcription analysis (WP 1); biometrics (WP 2); analysis of facial expressions & gestures (WP 3); eye-tracking (WP 4); and machine learning (WP5).

In a preferred embodiment, the process begins at Step 1, where the initial video is created. In Step 2, the recorded speech is converted to text before it can be used for other purposes. In Step 3, the converted, transcribed speech is transferred back to the video creator, while in Step 4, the transcription is stored, in particular, stored in a cloud service through a web service interface. In Step 5 and 6, the transcription is analyzed through a database to extract keywords; in Step 7, the database saves all detected key words and moments for future analysis. In Step 8, an algorithm starts biometric analysis, evaluating the unique physical and behavioral characteristics embedded in the video; a separate biometric software can be used (Step 9). In Step 10, the biometric data is saved in the database for future reference. In step 11, similar to the biometric analysis in Step 8 and 9, the algorithm includes gesture analysis. As shown under Step 12 and 13, the video is then transferred to a program to analyze different components of visual actions such as motion of hands, facial expression, and torso posture and movement. After the third-party gesture recognition software produces results for analysis, the results are stored in the database (Step 14). The same process is used for the eye tracking analysis (step 15), where the video is passed to the eye-tracking server (step 16) and stored in the database (step 17).

In further embodiments, the system can utilize: (i) AWS EC2: Production Servers; (ii) AWS S3: Storing Videos and Files; (iii) PHP, Yii2: programming language and framework; (iv) PostgreSQL: database; (v) AWS Transcribe: Automatic Transcription System; and (vi) TensorFlow: framework for AI development.

In FIG. 1, it is also exhibited the incorporation of artificial intelligence (“AI”) into the process (Step 18 & 19), as the results are placed in an AI system, with the module results saved in the database (step 20 & 21). In step 22, the final step, the results from the above are shown to the user through the interface.

The primary technologies used: (i) Cloud computing platform; (ii) Cloud storage: Storing Videos and Files; (iii) Backend programming language and framework; (iv) Database system; (v) Transcribe service: Automatic Transcription System; and (vi) Framework for AI development.

### WP 1: Transcription Analysis

The automated machine based transcription of the audio section of the recording is analyzed, and searched for keywords and key phrases that have been previously defined or added as a specific customization by a user, or incorporated and added by the AI algorithm and/or machine learning component. The system considers how many seconds or minutes before and after the word need to be taken into account, so as to understand the context. Thus, a total amount of time before and after the key moment is included, so that a reviewer, by clicking on the key moment, will start at X seconds before and continue to Y seconds after the key moment. At the developmental level, the same keywords referenced in prior tests are used to analyze existing or future transcripts. The algorithm also contains a search functionality to return a set of highlighted keywords. Example: For a 20-minute video, the keywords “error” and “confusion” are found. The word error is found at minute 5. The algorithm returns:
1) Word: “error”
2) Time: 00:05:00
3) Start: 00:04:30
4) End: 00:06:00

The word “confusion” is found at 10:07. Return:
1) Word: “confusion”
2) Time: 00:10:07
3) Start: 00:09:37
4) End: 00:11:07

The same happens with the sentences classified as positive and negative. This information is returned to a video player from which users are able to click on them so they can reproduce and focus only on those sections. Users are also able to modify the results if they consider it a false positive, neutral or negative.

### WP 2: Biometrics

Using smartwatches and other wearable hardware, where the hardware is placed on specific locations of the Tester’s body, where certain motions, movements, or actions are monitored, extracted, and eventually added to the system. Therefore, the system collects the participant’s relevant biofeedback information that is sent to servers to provide more data for the analysis described above. For example, the wearable is able to detect an increase in pulse rate at a certain time, and sends that information to the servers for combining with the semantic analysis described above with the e-ticket: “high pulsations”. Regardless of the specific devices used, the devices capture the data that corresponds with the user’s movements so that the data related to heart rates, temperature, motions, and other information about the user is collected.

The growing penetration in the market of “smartwatches” and similar tools that measure biological parameters, such as sweat, tension, pulse, skin temp etc., make it practicable to complement the semantic, facial, and eye tracking inputs described above with an analysis of the state of participants based on biological measurements that allow us to know their mood and degree of frustration and/or neurological effort while performing a certain task. Other “wearable” devices, such as headbands, embedded sensors, and trackers, are common examples of integrated gadgets to accurately quantify movement and collect data from the human body. Multiple gadgets can be used to assess the User’s functions and/or detect the Tester’s movement in the testing environment.

The connection between the wearable and the servers requires a specific app in the wearable. The user will be prompted with a code that, once introduced into the device, it will link it with the session. Once the device starts capturing insights and sends the information to the servers, the servers know to which session it belongs to.

That way, while the user is interacting with the asset, if the pulse is high or low for some time, the system collects the timestamp information. Then, the video player informs the system about those key moments. That information may be compared with the sentiment analysis to verify if the high pulse corresponds with specific keywords, so the system can add more related information about those instants.

The client as well as testing staff will be able to review and process the data, and correct the values if required, via the interface.

### WP 3: Analysis of Facial Expressions & Gestures

A “sentiment analysis” is an analysis of facial expressions as well as gestures. For sentiment analysis, the system sends related expression and gesture data to the servers, including timestamping the moments that relate to the emotional state of mind of the participant, and this information is added to the semantic analysis and biofeedback input described above.

As an example, if the system detects a certain facial gesture, it collects the timestamps; this is the same with other gestures.

The data is used to highlight those moments and compare with the other information collected in the previous stages, such as keywords and biometric information, adding more relevant information about those times.

### WP 4: Eye-Tracking

Data related to the direction of the individual’s gaze (“Eye-tracking”) is found in the recording and sent to the servers, incorporating the relevant timeline moments, so as to add gaze direction, facial expression, biofeedback, semantic analysis and the potential customizable “goals” of the user (e.g.: “...any gaze at asset Z is important...”) to the analysis. The data sent to the servers includes data similar to:
1) Minute 00:02:00 - 00:05:00
2) Centered in upper left area, with a 95% (or higher) degree of confidence

The system may save information about the user centering the view in one specific area, or, on the other hand, looking at different places quickly (for example if he/she is looking for something and is unable to locate it).

Beyond the initial calibration process, eye-tracking technology is usually complemented with mouse pointer position tracking and clicking, in order to improve accuracy of eyesight.

Counting/registering the number of clicks required to complete a particular task would be also useful to analyze/compare the difficulty level each user experienced to complete the task. Quantifying mouse movements/displacement during each task would be another interesting key to consider.

In the same way we are identifying some valuable keywords during semantic analysis, it should be possible to define some “hot-spots”, points of interest in the interface during the eye tracking process (A/B tests, etc.). The eyes of the user looking at one of these hot spots would be also marked as a moment of interest. Multiple and differently weighted hot spots may be defined.

As the other WPs, this data is included and compared with the rest of the information.

### WP 5: Machine Learning

Using a framework for machine learning and artificial intelligence, our AI engine feeds on all the information saved in the previous phases. This system is “taught” in the sense of detracting events that were not considered “interesting”, as well as adding those that were not identified by the system. From this, the engine can incorporate new important words or phrases, or combinations of words, phrases, gaze directions, facial expressions, and biofeedback that correlates with interesting events and comments.

The system automatically learns from the collected information. However, in order to give it more value, experts in UX may review the output of VideoAl, and when they detect a “false positive”, so indicate to the system. And when they detect that a “MOI” (Moment of Interest) is present, but not detected, so indicate and “feed” the machine learning system. This is allowed to the users in the interface.

These phases, through the process, are combined ... describe ... and an output is presented to a user interface. The user interface incorporates the video and the results obtained, with the option to immediately go to each of the key moments detected by the system, and listen/view them. A search option allows the keywords of the voice transcription to be analyzed, with its associated time stamp. The screen shows all the key points in time bar mode highlighting those minutes and with the option to click on each one to go to the exact moment in the video.

In a preferred embodiment, the system goes far beyond a simple analysis of phrases and words. Facial recognition has experienced a very considerable advance in recent years, allowing the most advanced systems to identify, with pinpoint accuracy, the identity of people. Even so, the identification of feelings, or moods, with some degree of precision, is still very incipient in facial analysis systems, and, without the additional layers of input brought to the analysis by the system of the present application cannot provide a increased accuracy of results.

In a preferred embodiment, the artificial intelligence system combines and integrates each of the data inputs, as well as automatic voice transcription, to, in an automated way, and with an accuracy that increases as more video sessions are analyzed over time via “machine learning,” detect and point out which moments in a series of recordings that can involve many hours of audiovisual content, are the most important to review, listen and see.

Moreover, the system can be customized for each client and each “use case” in such a way that a client, for a specific project, can “program” the system with the objectives and research goals pursued, and receive, instantly, or almost instantaneously, the signaling of the important moments to visualize and listen following these programmed and customizable parameters in real time.

It should be noted that, although the embodiments being discussed are in context of the specific use of automated analysis of user experience and market research data, it can nonetheless be applied to a diverse set of applications from many very diverse fields, such as: (i) analysis of acceptance testing of advertising videos or movies or movie trailers; (ii) analysis of “Focus Group” recordings; (iii) analysis of audiovisual recordings of webinars, conferences and the like; (iv) quick location of the most interesting moments to consume, by users of audiovisual social networks similar to YouTube; and (v) determine specific moments, feelings, desires of people with reduced mobility and/or communication problems.

Any of the data provided by the modules can be edited by the client or testing staff, so the AI will learn about the corrections. Once all data is available, the client or testing staff reviews them and applies the required changes, which will feed the AI with more learning information for the future. The interface provides the capability to change the results.

1. A method for analyzing qualitative remote user experience and usability test results using artificial intelligence, the method comprising:
selecting at least one participant based on predetermined criteria;
recording data of the at least one participant’s interaction with a test session through remote testing software;
inputting the recorded data from the test session into a central computer for data analysis;
identifying a plurality of moments of interests of the participant’s interaction from the test session by synthesizing semantic data, eye tracking data, biosensor input data, and facial analysis from the inputted recorded data;
training the artificial intelligence by
classifying at least one identified moment of interest as a detracting event,
classifying at least one non-identified moment of interest as a moment of interest,
identifying which input data is associated with the detracting events,
identifying which input data is associated with the non-identified moments of interest, and
identifying which input data is associated with moments of interest,
outputting the recorded data of the participant’s interaction with the test session with the identified moments of interest.

2. The method according to claim 1, wherein the recorded data is a video recording of the participant’s screen during the participant’s interaction with the test session, an audiovisual recording of the participant’s interaction with the test session, and/or biosensor data from a biosensor device worm by the participant during the test session.

3. The method according to claim 2, wherein the recorded data is outputted to a user interface with the video recording of the participant’s screen and/or the audiovisual recording of the participant having identified moments of interest timestamped.

4. The method according to claim 1, further comprising:
identifying data sets that are associated with multiple detracting events; and
identifying data sets that are associated with multiple non-identified moments of interest; and
identifying data sets that are associated with multiple moments of interest.

5. The method according to claim 1, further comprising:
identifying data sets that are associated with at least one detracting event;
identifying data sets that are associated with at least one non-identified moment of interest; and
identifying data sets that are associated with at least one moment of interest.

6. The method according to claim 1, wherein,
the recorded data is input into the central computer during the at least one participant’s interaction with the test session;
identifying at least one moment of interest during the at least one participant’s interaction with the test session;
training the artificial intelligence during the at least one participant’s interaction with the test session; and
identifying, thereafter, at least one further moment of interest.
